(19) United States
(12) Patent Application Publication
(10) Pub. No.: US 2025/0143981 A1
(43) Pub. Date: May 8, 2025

(54) AEROSOL DEVICE CONTAINING A COMPOSITION COMPRISING A BRANCHED ALKANE, A FATTY SUBSTANCE AND A PROPELLANT

(71) Applicant: L'OREAL, Paris (FR)

(72) Inventors: Lionel AUBERT, SAINT-OUEN (FR); Nadia SMAIL, SAINT-OUEN (FR); Raquel STEFANI, SAINT-OUEN (FR); Carine AIRES, SAINT-OUEN (FR)

(21) Appl. No.: 18/833,119

(22) PCT Filed: Jan. 24, 2023
(86) PCT No.: PCT/EP2023/051710

§ 371 Date: Jul. 25, 2024

(30) Foreign Application Priority Data
Jan. 28, 2022 (FR) FR2200779

(51) Int. Cl. A61K 8/04; A45D 19/00; A45D 34/04; A61K 8/31; A61K 8/892; A61Q 5/12; B65D 83/141; B65D 83/20; B65D 83/30

(52) U.S. Cl. CPC A61K 8/046; A45D 19/0041; A45D 34/04; A61K 8/31; A61K 8/892; A61Q 5/12; B65D 83/141; B65D 83/20; B65D 83/30

(57) ABSTRACT

The invention relates to an aerosol device comprising:
a container containing a composition comprising an amount greater than or equal to 30% by weight relative to the total weight of the composition of one or more C6-C14 branched alkanes, one or more fatty substances other than the C6-C14 branched alkanes and one or more propellants,
a means for dispensing said composition, including:
   a body (3) that is open at its two opposite axial ends,
   an engaging part (10) that is open at its two opposite axial ends, at least partially defining at least one dispensing orifice (12).

The present invention relates to an aerosol device comprising a particular dispensing means and a composition comprising at least 30% by weight of one or more C6-C14 branched alkanes, at least one additional fatty substance and at least one propellant.

The invention also relates to a process for conditioning keratin fibers, in particular human keratin fibers such as the hair, comprising at least one step of applying the composition according to the invention to said fibers.

The invention lastly relates to the use of the composition according to the invention sprayed from the particular aerosol device for conditioning keratin fibers, in particular human keratin fibers such as the hair, and notably for providing softness, a smooth, non-tacky feel and sheen, for facilitating the disentangling of the hair, for defining the hair curls, and for providing control of the frizziness and volume of the head of hair. The device associated with the composition makes it possible to deliver a light mist, which does not weigh down the hair and is easy to spread on the hair without a greasy effect.

Many people find that their hair takes too long to dry, but the use of heating tools can sensitize the hair (curly, damaged, fine, long, etc. hair)

They are also seeking to give their hair conditioning properties without having negative effects such as a greasy look or feel.

Indeed, these people notably want their hair to be easy to disentangle, and to be soft and shiny.

Moreover, people with curly hair may want better curl definition, better uniformity, and less volume and frizziness, even in humid conditions. Thus, some people find it difficult to control, define and/or style their curly hair.

There is a need to provide compositions that simplify the styling routine, decrease the drying time of the hair, provide conditioning to the hair and protect the hair from heat.

The Applicant has found, surprisingly and advantageously, that using a device equipped with a dispensing means including a body open at its two opposite axial ends and an engaging part open at its two opposite axial ends, at least partially defining a dispensing orifice, for dispensing a composition comprising at least 30% by weight of one or more C6-C14 branched alkanes, one or more fatty substances, other than the C6-C14 branched alkanes, and at least one propellant, makes it possible to obtain better dispensing of the composition, a more homogeneous result, faster drying, and a soft, smooth and non-greasy feel, without transfer and without a wet appearance. The hair is shiny and visually smooth with frizz control.

According to a first of its aspects, a subject of the invention is an aerosol device comprising:
a container containing a composition comprising an amount greater than or equal to 30% by weight relative to the total weight of the composition of one or more C6-C14 branched alkanes, one or more substances other than the C6-C14 branched alkanes and one or more propellants,
a means for dispensing said composition, including:
   a body open at its two opposite axial ends,
   an engaging part open at its two opposite axial ends, at least partially defining at least one dispensing orifice.

This particular combination allows easy application and homogeneous, fine and light dispensing of the hair composition on the head of hair, thus leading to improved conditioning properties with a natural result.

It enables accelerated drying of the hair with or without an input of heat. It in particular affords a pleasant cosmetic feel, notably a smooth, non-tacky feel, without transfer, it facilitates the disentangling of the hair and provides improved sheen. The composition does not give a greasy effect and affords volume control.

The present invention also relates to a process for conditioning keratin fibers, comprising the use of the device as defined previously. In particular, the process comprises a step of applying to wet or dry hair, preferably wet hair, a composition sprayed from an aerosol device according to the invention, optionally a rinsing step, preferably without rinsing after an optional leave-on time.

Other subjects, characteristics, aspects and advantages of the invention will emerge even more clearly on reading the description and the example that follows.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

The term “keratin fibers” means fibers of human or animal origin, such as head hair, bodily hair, the eyelashes, the eyebrows, wool, angora, cashmere or fur. According to the present invention, the keratin fibers are preferably human keratin fibers, more preferentially the hair.

According to the invention, the aerosol device comprises a container which contains a composition comprising an amount greater than or equal to 30% by weight of one or more C6-C14 branched alkanes, one or more fatty substances other than the C6-C14 branched alkanes and at least one propellant.

### C6-C14 Branched Alkanes

The C6-C14 branched alkane(s) are preferably C8-C14 branched alkanes. They are notably chosen from isohexane, isooctane, isodecane, isododecane and mixtures thereof. Isododecane is particularly preferred.

Preferably, the C6-C14 branched alkane(s) are present in the composition in an amount ranging from 30% to 70% by weight, better still from 40% to 60% by weight, and even more preferentially from 45% to 55% by weight relative to the total weight of the composition.

### Propellants

The composition according to the invention also comprises one or more propellants.

Examples of propellants that may be used in the aerosol device of the present invention are liquefied gases such as dimethyl ether, chlorinated and/or fluorinated hydrocarbons such as 1,1-difluoroethane, or volatile hydrocarbons notably such as C3-5 alkanes, for instance propane, isopropane, n-butane, isobutane or pentane, or compressed gases such as air, nitrogen or carbon dioxide, and mixtures thereof.

Mention may be made preferentially of dimethyl ether and C3-5 alkanes and in particular propane, n-butane and isobutane, and mixtures thereof. More preferentially, the propellant is isobutane.

Preferably, the propellant(s) are present in the composition in an amount ranging from 30% to 70% by weight, better still from 40% to 60% by weight, and even more preferentially from 45% to 55% by weight relative to the total weight of the composition. Fatty substance

The composition also comprises one or more fatty substances other than the C6-C14 branched alkanes.

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by the company TA Instruments. In the present patent application, all the melting points are determined at atmospheric pressure (1.013×10⁵ Pa).

The term “fatty substance” means an organic compound that is insoluble in water at 25° C. and at atmospheric pressure (1.013×10⁵ Pa) (solubility of less than 5% by weight, preferably less than 1% by weight and even more preferentially less than 0.1% by weight). The fatty substances have in their structure at least one hydrocarbon-based chain including at least six carbon atoms and/or a sequence of at least one siloxane group. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The fatty substances other than the C6-C14 branched alkanes may be solid or liquid, and silicone-based or not silicone-based.

The term “fatty ester” means an ester of a fatty acid and/or of a fatty alcohol.

The term “fatty ether” means a compound comprising at least one linear or branched, saturated or unsaturated ether function, comprising at least one hydrocarbon-based group containing from 6 to 40 carbon atoms.

In particular, the fatty substances of the invention are not (poly)oxyalkylenated or (poly)glycerolated ethers.

The term “oil” means a “fatty substance” that is liquid at room temperature (25° C.) and at atmospheric pressure (760 mmHg or 1.013×10⁵ Pa).

The term “non-silicone fatty substance” means a compound which does not contain a sequence of siloxane (—SiO—) groups, preferably which does not contain any silicon atoms.

### Solid Fatty Substances

The solid fatty substances generally have a melting point of greater than 25° C., preferably greater than or equal to 28° C., more preferentially greater than or equal to 30° C. at atmospheric pressure (1.013×10⁵ Pa). In the present patent application, this or these fatty substances are also referred to as “solid fatty substance(s)”.

The solid fatty substances according to the invention preferably have a viscosity of greater than 2 Pa·s, measured at 25° C. and at a shear rate of 1 s⁻¹.

The fatty substance(s) with a melting point of greater than 25° C. are preferably chosen from solid fatty acids, solid fatty alcohols, solid esters of fatty acids and/or of fatty alcohols, waxes, butters, ceramides, solid monoglycerides, diglycerides or triglycerides, and mixtures thereof.

The term “fatty acid” means a long-chain carboxylic acid comprising from 6 to 40 carbon atoms, preferably from 8 to 30 carbon atoms. The solid fatty acids according to the invention preferentially comprise from 10 to 30 carbon atoms and better still from 14 to 22 carbon atoms. These fatty acids are neither oxyalkylenated nor glycerolated.

The solid fatty acids that can be used in the present invention are notably chosen from myristic acid, cetylic acid, stearylic acid, palmitic acid, stearic acid, lauric acid, behenic acid, and mixtures thereof.

The term “fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated.

The solid fatty alcohols may be saturated or unsaturated, and linear or branched, and include from 8 to 40 carbon atoms, preferably from 10 to 30 carbon atoms, better still from 12 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon 15 atoms, better still from 12 to 30, or even from 12 to 24 atoms and even better still from 14 to 22 carbon atoms.

The solid fatty alcohols that may be used are preferably chosen from saturated, and linear or branched, preferably linear and saturated, (mono) alcohols including from 8 to 40 carbon atoms, better still from 10 to 30, or even from 12 to 24 atoms and better still from 14 to 22 carbon atoms.

The solid fatty alcohols that may be used may be chosen, alone or as a mixture, from:
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl or cetearyl alcohol. Particularly preferably, the solid fatty alcohol is chosen from cetyl alcohol, stearyl alcohol or mixtures thereof such as cetylstearyl alcohol, better still the solid fatty alcohol is cetylstearyl alcohol.

The solid esters of a fatty acid and/or of a fatty alcohol that may be used are preferably chosen from esters derived from a C₉-C₂₆ carboxylic fatty acid and/or from a C₉-C₂₆ fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol, including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may notably be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C₉-C₂₆ alkyl palmitates, notably myristyl palmitate, cetyl palmitate and stearyl palmitate; C₉-C₂₆ alkyl myristates, such as cetyl myristate, stearyl myristate and myristyl myristate; C₉-C₂₆ alkyl stearates, notably myristyl stearate, cetyl stearate and stearyl stearate; and mixtures thereof.

Particularly preferably, the solid esters of a fatty acid and/or of a fatty alcohol are chosen from myristyl stearate, myristyl palmitate, and mixtures thereof.

For the purposes of the present invention, a wax is a lipophilic compound, which is solid at 25° C. and atmospheric pressure, with a reversible solid/liquid change of state, having a melting point greater than about 40° C., which may be up to 200° C., and having in the solid state anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal, plant or mineral origin, nonsilicone synthetic waxes, and mixtures thereof.

Mention may notably be made of hydrocarbon-based waxes, for instance beeswax or modified beeswaxes (cera bellina), lanolin wax and lanolin derivatives, spermaceti; cork fiber or sugarcane waxes, olive tree wax, rice bran wax, carnauba wax, candelilla wax, ouricury wax, esparto grass wax, berry wax, shellac wax, Japan wax and sumac wax, absolute waxes of flowers; montan wax, orange wax, lemon wax, microcrystalline waxes, paraffins, petroleum jelly, lignite and ozokerite; polyethylene waxes, the waxes obtained by Fischer-Tropsch synthesis and waxy copolymers, and also esters thereof.

Mention may also be made of C2 to C60 microcrystalline waxes, such as Microwax HW.

Mention may also be made of the PM 500 polyethylene wax sold under the reference Permalen 50-L polyethylene.

Mention may also be made of the waxes obtained by catalytic hydrogenation of animal or plant oils containing linear or branched C8 to C32 fatty chains. Among these waxes, mention may notably be made of isomerized jojoba oil such as trans-isomerized partially hydrogenated jojoba oil, notably the product manufactured or sold by the company Desert Whale under the commercial reference Iso-Jojoba-50®, hydrogenated sunflower oil, hydrogenated castor oil, hydrogenated coconut kernel oil, hydrogenated lanolin oil and bis(1,1,1-trimethylolpropane)tetrastearate, notably the product sold under the name Hest 2T-4S® by the company Heterene.

The waxes obtained by hydrogenation of castor oil esterified with cetyl alcohol, such as those sold under the names Phytowax Castor 16L64® and 22L73® by the company Sophim, may also be used.

A wax that may be also used is a C20 to C40 alkyl (hydroxystearyloxy) stearate (the alkyl group comprising from 20 to 40 carbon atoms), alone or as a mixture. Such a wax is notably sold under the names Kester Wax K 82 P®, Hydroxypolyester K 82 P® and Kester Wax K 80 P® by the company Koster Keunen.

It is also possible to use microwaxes in the compositions of the invention; mention may notably be made of carnauba microwaxes, such as the product sold under the name MicroCare 3500 by the company Micro Powders, synthetic-wax microwaxes, such as the product sold under the name MicroEase 114S® by the company Micro Powders, microwaxes constituted of a mixture of carnauba wax and polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 2200, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin, petroleum jelly, lignite or ozokerite wax; plant waxes, for instance cocoa butter, shea butter or cork fiber or sugar cane waxes, olive tree wax, rice bran wax, hydrogenated jojoba wax, ouricury wax, carnauba wax, candelilla wax, esparto grass wax, or absolute waxes of flowers, such as the essential wax of blackcurrant blossom sold by the company Bertin (France); waxes of animal origin, for instance beeswaxes or modified beeswaxes (cera bellina), spermaceti, lanolin wax and lanolin derivatives; microcrystalline waxes; and mixtures thereof.

Butters may also be used.

For the purposes of the present invention, the term “butter” (also referred to as “pasty fatty substance”) means a lipophilic fatty compound with a reversible solid/liquid change of state and including, at a temperature of 25° C. and at atmospheric pressure (760 mmHg), a liquid fraction and a solid fraction. Preferably, the butter(s) according to the invention have a melting start temperature above 25° C. and a melting end temperature below 60° C.

Preferably, the particular butter(s) are of plant origin, such as those described in Ullmann's Encyclopedia of Industrial Chemistry (“Fats and Fatty Oils”, A. Thomas, published online: Jun. 15, 2000, DOI: 10.1002/14356007.a10_173, point 13.2.2.2. Shea Butter, Borneo Tallow, and Related Fats (Vegetable Butters)).

Mention may be made more particularly of shea butter, Nilotica shea butter (Butyrospermum parkii), galam butter (Butyrospermum parkii), Borneo butter or fat or tengkawang tallow (Shorea stenoptera), shorea butter, illipé butter, madhuca butter or Bassia madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya butter (Kpangnan) (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter and sunflower butter.

The ceramides, or ceramide analogs such as glycoceramides, which may be used in the compositions according to the invention, are known; mention may be made in particular of ceramides of classes I, II, III and V according to the Dawning classification.

The ceramides or analogs thereof that may be used preferably correspond to the following formula:

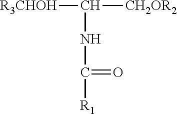

in which:
R₁ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R₂ denotes a hydrogen atom, a (glycosyl) n group, a (galactosyl) m group or a sulfogalactosyl group, in which n is an integer ranging from 1 to 4 and m is an integer ranging from 1 to 8;
R₃ denotes a C₁₅-C₂₆ hydrocarbon-based group, which is saturated or unsaturated in the alpha position, it being possible for this group to be substituted with one or more C₁-C₁₄ alkyl groups;

it being understood that, in the case of natural ceramides or glycoceramides, R₃ may also denote a C₁₅-C₂₆ alpha-hydroxyalkyl group, the hydroxyl group optionally being esterified with a C₁₆-C₃₀ alpha-hydroxy acid.

Preferentially, use is made of ceramides for which R₁ denotes a saturated or unsaturated alkyl group derived from C₁₄-C₃₀ fatty acids; R₂ denotes a galactosyl or sulfogalactosyl group; and R₃ denotes a —CH═CH—(CH₂) ₁₂—CH₃ group.

The ceramides that are more particularly preferred are the compounds for which R₁ denotes a saturated or unsaturated alkyl derived from C16-C22 fatty acids; R₂ denotes a hydrogen atom and R₃ denotes a saturated or unsaturated linear C15 group.

Use may also be made of the compounds for which R₁ denotes a saturated or unsaturated alkyl radical derived from C₁₂-C₂₂ fatty acids; R₂ denotes a galactosyl or sulfogalactosyl radical and R₃ denotes a saturated or unsaturated C₁₂-C₂₂ hydrocarbon-based radical and preferably a —CH═CH—(CH₂) ₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in N-particular stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl) malonamide; and mixtures thereof. N-Oleoyldihydrosphingosine will preferably be used.

Preferably, the fatty substance(s) with a melting point of greater than 25° C. are chosen from solid fatty alcohols, solid esters of fatty acids and/or of fatty alcohols, waxes, butters, and mixtures thereof, more preferentially from solid fatty alcohols, solid esters of fatty acids and/or of fatty alcohols, butters, and mixtures thereof.

The liquid fatty substances other than the C6-C14 branched alkanes may be chosen from triglycerides of plant or synthetic origin, fluoro oils, fatty esters other than triglycerides, liquid C₆ to C₁₆ hydrocarbons, liquid hydrocarbons comprising more than 16 carbon atoms, fatty alcohols, fatty ethers, dialkyl carbonates, silicones and mixtures thereof.

The liquid fatty substances other than the C6-C14 branched alkanes are preferably chosen from triglycerides of plant or synthetic origin, fluorinated oils, liquid fatty esters other than triglycerides, liquid fatty alcohols, liquid fatty ethers, dialkyl carbonates, silicones and mixtures thereof.

More particularly, the liquid fatty substances other than the C6-C14 alkanes are chosen from liquid esters of fatty acid and/or of fatty alcohol and silicones, and mixtures thereof.

### Triglycerides

The triglyceride oils of plant or synthetic origin are preferably chosen from liquid triglycerides of fatty acids including from 6 to 30 carbon atoms, such as heptanoic or octanoic acid triglycerides, or else, for example, sunflower oil, linseed oil, olive oil, safflower oil, argan oil, maize germ oil, wheat germ oil, soybean oil, marrow seed oil, grapeseed oil, sesame oil, hazelnut oil, apricot kernel oil, macadamia oil, arara oil, castor oil, avocado oil, caprylic/capric acid triglycerides, for instance those sold by the company Stéarinerie Dubois or those sold under the names Miglyol® 810, 812 and 818 by the company Dynamit Nobel, shea butter oil, and mixtures thereof.

### Liquid Fatty Alcohols

The liquid fatty alcohols that are suitable for use in the invention are more particularly chosen from linear or branched, saturated or unsaturated alcohols, preferably unsaturated or branched alcohols, including from 6 to 40 carbon atoms and preferably from 8 to 30 carbon atoms. Examples that may be mentioned include octyldodecanol, 2-butyloctanol, 2-hexyldecanol, 2-undecylpentadecanol, isostearyl alcohol, isocetyl alcohol, oleyl alcohol, linolenyl alcohol, ricinoleyl alcohol, undecylenyl alcohol and linoleyl alcohol, and mixtures thereof.

### Liquid Fatty Esters

As regards the liquid esters of fatty acids and/or of fatty alcohols other than the triglycerides mentioned previously, mention may notably be made of esters of saturated or unsaturated, linear C₁-C₂₆ or branched C₃-C₂₆ aliphatic monoacids or polyacids and of saturated or unsaturated, linear C₁-C₂₆ or branched C₃-C₂₆ aliphatic monoalcohols or polyalcohols, the total carbon number of the esters being greater than or equal to ₁₀.

Preferably, for the esters of monoalcohols, one at least of the alcohol or of the acid from which the esters of the invention are derived is branched and/or unsaturated.

Among the monoesters, mention may be made of dihydroabietyl behenate; octyldodecyl behenate; isocetyl behenate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl palmitate, isopropyl palmitate, 2-ethylhexyl palmitate, 2-octyldecyl palmitate, alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of isopropyl palmitates, alkyl myristates such as isopropyl myristate, isocetyl stearate, 2-ethylhexyl isononanoate, isodecyl neopentanoate, isononyl isononanoate, and mixtures thereof. Still within the context of this variant, use may also be made of esters of C₄-C₂₂ dicarboxylic or tricarboxylic acids and of C₁-C₂₂ alcohols, and esters of monocarboxylic, dicarboxylic or tricarboxylic acids and of C₂-C₂₆ dihydroxy, trihydroxy, tetrahydroxy or pentahydroxy alcohols.

Mention may notably be made of: diethyl sebacate; diisopropyl sebacate; diisopropyl adipate; di-n-propyl adipate; dioctyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; glyceryl trilactate; glyceryl trioctanoate; propylene glycol dioctanoate; neopentyl glycol diheptanoate; diethylene glycol diisononanoate; and mixtures thereof.

The composition may also comprise, as fatty ester, esters of aromatic acids and C8-C18 fatty alcohols, such as esters of benzoic acid and C8-C16 fatty alcohols, notably C12-C15 alkyl benzoate.

The composition may also comprise, as fatty ester, sugar esters and diesters of C₆-C₃₀ and preferably C₁₂-C₂₂ fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may notably be chosen from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates and arachidonates, or mixtures thereof such as, notably, oleopalmitate, oleostearate and palmitostearate mixed esters.

More particularly, use is made of monoesters and diesters and notably sucrose, glucose or methylglucose mono- or di-oleates, -stearates, -behen -ates, -oleopalmitates, -linoleates, -linolenates and -oleostearates, and mixtures thereof.

An example that may be mentioned is the product sold under the name Glucate® DO by the company Amerchol, which is a methylglucose dioleate.

### Dialkyl Carbonates

Use may also be made of dialkyl carbonates, in particular di(C1-C12)alkyl carbonates, preferably di(C4-C12)alkyl carbonates notably such as dicaprylyl carbonate.

### Fatty Ethers

The liquid fatty ethers may be chosen from dialkyl ethers, in particular di(C1-C12)alkyl ethers, preferably di(C4-C12)alkyl ethers, notably such as dicaprylyl ether.

### Liquid Hydrocarbons

The liquid hydrocarbons other than the C6-C14 alkanes may be linear, optionally cyclic, and preferably are chosen from alkanes. Examples that may be mentioned include hexane, cyclohexane, undecane, dodecane, tridecane or isoparaffins, such as isohexadecane, and mixtures thereof.

The liquid hydrocarbons comprising more than 16 carbon atoms may be linear or branched, and of mineral or synthetic origin, and are preferably chosen from liquid paraffins or liquid petroleum jelly, polydecenes, hydrogenated polyisobutene such as Parleam®, and mixtures thereof.

### Silicones

The silicones that may be used in the cosmetic compositions of the present invention are volatile or non-volatile, cyclic, linear or branched silicones, which are unmodified or modified with organic groups, having a viscosity of 5×10⁻⁶ to 2.5 m²/s at 25° C., and preferably 1×10⁻⁵ to 1 m²/s.
The silicones that may be used in accordance with the invention may be in the form of oils, waxes, resins or gums.
Preferably, the silicone is chosen from polydialkylsiloxanes, in particular polydimethylsiloxanes (PDMSs), and organomodified polysiloxanes comprising at least one functional group chosen from poly(oxyalkylene) groups, amino groups and alkoxy groups.
Organopolysiloxanes are defined in greater detail in Walter Noll's Chemistry and Technology of Silicones (1968), Academic Press. They may be volatile or nonvolatile. When they are volatile, the silicones are more particularly chosen from those with a boiling point of between 60° C. and 260° C., and even more particularly from:

(i) cyclic polydialkylsiloxanes including from 3 to 7 and preferably from 4 to 5 silicon atoms. These are, for example, octamethylcyclotetrasiloxane sold notably under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Volatile Silicone® FZ 3109 sold by Union Carbide, having the formula:

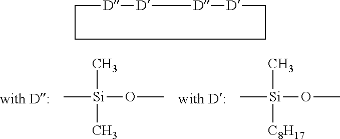

Mention may also be made of mixtures of cyclic polydialkylsiloxanes with organosilicon compounds, such as the mixture of octamethylcyclotetrasiloxane and tetra(trimethylsilyl) pentaerythritol (50/50) and the mixture of octamethylcyclotetrasiloxane and oxy-1,1′-bis(2,2,2′,2′,3,3′-hexatrimethylsilyloxy) neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane notably sold under the name SH 200 by the company Toray Silicone. Silicones falling within this category are also described in the article published in Cosmetics and Toiletries, Vol. 91, January 76, pages 27-32-Todd & Byers Volatile Silicone

### Fluids for Cosmetics.

Nonvolatile polydialkylsiloxanes, polydialkylsiloxane gums and resins, polyorganosiloxanes modified with the above organofunctional groups, and mixtures thereof, are preferably used.
These silicones are more particularly chosen from polydialkylsiloxanes, among which mention may be made mainly of polydimethylsiloxanes bearing trimethylsilyl end groups. The viscosity of the silicones is measured at 25° C. according to the standard ASTM 445 Appendix C.
Mention may be made, among these polydialkylsiloxanes, in a nonlimiting way, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.
   Mention may also be made of polydimethylsiloxanes bearing dimethylsilanol end groups, known under the name dimethiconol (CTFA), such as the oils of the 48 series from the company Rhodia.
   In this category of polydialkylsiloxanes, mention may also be made of the products sold under the names Abil Wax® 9800 and 9801 by the company Goldschmidt, which are polydi(C₁-C₂₀)alkylsiloxanes.
   The silicone gums that may be used in accordance with the invention are notably polydialkylsiloxanes and preferably polydimethylsiloxanes with high number-average molecular masses of between 200 000 and 1 000 000, used alone or as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.
   Products that may be used more particularly in accordance with the invention are mixtures such as:
mixtures formed from a polydimethylsiloxane with a hydroxy-terminated chain, or dimethiconol (CTFA), and from a cyclic polydimethylsiloxane, also known as cyclomethicone (CTFA), such as the product Q21401 sold by the company Dow Corning;
mixtures of a polydimethylsiloxane gum and a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs with different viscosities, and more particularly of a PDMS gum and a PDMS oil, such as the product SF 1236 from the company General Electric. The product SF 1236 is the mixture of a gum SE 30 defined above with a viscosity of 20 m²/s and of an oil SF 96 with a viscosity of 5×10⁻⁶ m²/s. This product preferably includes 15% of gum SE 30 and 85% of an oil SF 96.
   The organopolysiloxane resins that may be used in accordance with the invention are crosslinked siloxane systems containing the following units:
R₂SiO_{2/2}, R₃SiO_{1/2}, RSiO_{3/2} and SiO_{4/2},
in which R represents an alkyl containing 1 to 16 carbon atoms. Among these products, the ones that are particularly preferred are those in which R denotes a C₁-C₄ lower alkyl group, more particularly methyl.
   Among these resins, mention may be made of the product sold under the name Dow Corning 593 or those sold under the names Silicone Fluid SS 4230 and SS 4267 by the company General Electric, which are silicones of dimethyl/trimethylsiloxane structure.
   Mention may also be made of the trimethyl siloxysilicate-type resins notably sold under the names X22-4914, X21-5034 and X21-5037 by the company Shin-Etsu.
   The organomodified silicones that may be used in accordance with the invention are silicones as defined previously and including in their structure one or more organofunctional groups attached via a hydrocarbon-based group.
   In addition to the silicones described above, the organomodified silicones can be polydiarylsiloxanes, in particular polydiphenylsiloxanes, and polyalkylarylsiloxanes functionalized by the abovementioned organofunctional groups.
   The polyalkylarylsiloxanes are chosen in particular from linear and/or branched polydimethyl/methylphenylsiloxanes and polydimethyl/diphenylsiloxanes with a viscosity ranging from 1×10⁻⁵ to 5×10⁻² m²/s at 25° C.
   Among these polyalkylarylsiloxanes, examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.
   Among the organomodified silicones, mention may be made of polyorganosiloxanes including:
polyethyleneoxy and/or polypropyleneoxy groups optionally including C6-C24 alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;
substituted or unsubstituted amine groups, such as the products sold under the names GP 4 Silicone Fluid and GP 7100 by the company Genesee or the products sold under the names Q2 8220 and Dow Corning 929 or 939 by the company Dow Corning. The substituted amino groups are, in particular, C₁-C₄ aminoalkyl groups;
alkoxylated groups, such as the product sold under the name Silicone Copolymer F-755 by SWS Silicones and Abil Wax® 2428, 2434 and 2440 by the company Goldschmidt.

The silicones are preferably chosen from polydimethylsiloxanes (dimethicone), polydimethylsiloxanes bearing dimethyl silanol (dimethiconol) end groups, phenyl silicones such as phenyl trimethicones, phenylmethicone dimethicones, phenyl trimethylsiloxy diphenyl siloxanes, diphenyl dimethicones, and diphenyl methyldiphenyl trisiloxanes, preferably from polydimethylsiloxanes (dimethicone), polydimethylsiloxanes bearing dimethyl silanol (dimethiconol) end groups, and mixtures thereof.

Preferably, the fatty substance(s) other than the C6-C14 branched alkanes are chosen from nonsilicone organic liquid fatty substances and silicones and mixtures thereof.

Preferably, the fatty substance(s) other than the C6-C14 branched alkanes are chosen from liquid fatty esters, silicones, and mixtures thereof, preferentially from esters of aromatic acids and of C8-C18 fatty alcohols, silicones bearing dimethyl silanol end groups (dimethiconol), and mixtures thereof.

The composition according to the invention may comprise the fatty substance(s) other than the C6-C14 branched alkanes in a total content ranging from 0.1% to 15% by weight, preferably from 0.5% to 10% by weight, preferentially from 1% to 5% by weight, relative to the total weight of the composition.

The composition according to the invention may comprise the organic liquid fatty substance(s) and silicones other than the C6-C14 branched alkanes in a total content ranging from 0.1% to 15% by weight, preferably from 0.5% to 10% by weight, preferentially from 1% to 5% by weight, relative to the total weight of the composition.

The composition may also comprise one or more C₂-C₄ monoalcohols.

As C₂-C₄ monoalcohol(s) that may be used in the aerosol device of the invention, mention may notably be made of ethanol or isopropanol, or better still ethanol.

When they are present, the C₂-C₄ monoalcohol(s) are preferably present in an amount of less than 5% by weight, better still less than 2% by weight relative to the total weight of the composition.

Preferably, the composition does not comprise any C₂-C₄ monoalcohols.

The composition according to the invention may contain one or more additional organic solvents such as polyols, for instance 1,2-propylene glycol, 1,3-propanediol, 1,3-butylene glycol, hexylene glycol, diethylene glycol, dipropylene glycol, 2-ethoxyethanol, triethylene glycol monomethyl ether and sorbitol. Propylene glycol and glycerol will be used more particularly.

When they are present, the organic solvent(s) are preferably present in an amount of less than 5% by weight, relative to the total weight of the composition, in particular in a total content ranging from 0.01% to 5% by weight, preferably from 0.1% to 3% by weight, relative to the total weight of the composition.

The composition may also contain water.

Preferably, the composition according to the invention contains less than 5% by weight and preferably less than 1% by weight of water relative to the total weight of the composition. Even more preferentially, the composition does not contain any added water. The composition is then said to be anhydrous.

The compositions defined in the invention may also contain one or more additives chosen from anionic, cationic, nonionic, amphoteric or zwitterionic fixing and/or conditioning polymers, fragrances, dyes, protective UV-screening agents, acids, bases, nacres, glitter flakes, absorbent and/or styling powders and mixtures thereof.

These additives may be present in the composition according to the invention in an amount ranging from 0 to 20% by weight, relative to the total weight of the composition.

A person skilled in the art will take care to select these optional additives and the amounts thereof such that they do not harm the properties of the compositions of the present invention.

A subject of the present invention is also a process for conditioning keratin fibers, in particular human keratin fibers such as the hair, comprising at least one step of applying to said fibers a composition as described previously.

Preferably, the composition according to the invention is applied to wet or dry hair and preferably to wet hair.

This or these steps of applying the composition according to the invention may optionally be preceded by at least one step of washing said fibers using a shampoo.

Moreover, this or these steps of applying the composition according to the invention may optionally be followed by a step of leaving the composition on, for example a leave-on time of 5 s to 15 minutes, notably 15 s to 5 minutes; then followed by an optional rinsing step, for example with water; and/or a drying step.

Preferably, the keratin fibers are not rinsed after the step(s) of applying the composition according to the invention. The expression “are not rinsed” means that no step of rinsing the keratin fibers is performed within a period of less than 30 minutes, preferentially of 20 minutes, after the step of applying the composition according to the invention.

Preferably, the invention is a process using the device comprising a composition as described above to decrease the drying time of wet hair.

### Aerosol Device

The compositions in accordance with the invention are packaged in an aerosol device comprising a container, also called a reservoir, and a particular dispensing means.

The container is pressurized and comprises the composition to be dispensed. The container containing the pressurized composition may be opaque or transparent. It may be made of glass, polymer or metal, optionally coated with a protective varnish coat.

The container is equipped at its top end with a valve that seals the system.

Onto this valve is fitted a dispensing means, on which the user can press to make the product come out. This dispensing means is also known as a diffuser.

As indicated previously, the dispensing means according to the invention includes a body open at its two opposite axial ends and an engaging part open at its two opposite axial ends, at least partially defining at least one dispensing orifice.

In particular, the dispensing orifice is preferably defined between the body and the engaging part but may alternatively be defined entirely by the engaging part.

By means of the device of the invention, a passage is provided through the dispensing means and more particularly through the body and the engaging part, allowing a circulation of air to be established through the dispensing means when the product to be dispensed is emitted, which may be advantageous when the product is emitted in the form of a spray, by allowing a flow of air to be created through the dispensing means to accompany the flow of the spray.

In addition, the passage through the dispensing means can be made with sufficient dimensions to allow, if so desired, a finger or a lock of hair to be inserted into this passage. This can facilitate the application of a product to the finger or lock of hair.

If so desired, the invention may also make it easier to produce a dispensing orifice having an annular section between the engaging part and the body, allowing the formation of a hollow spray. Alternatively, a plurality of dispensing orifices are formed between the body and the engaging part, for example so as to dispense the product in the form of a number of sprays or jets. The number of dispensing orifices may notably be between 2 and 80, preferably between 5 and 60, and better still between 5 and 20, limits inclusive. It may, for example, be equal to 10. The dispensing orifices each have, for example, a cross section of greater than or equal to 0.0025 mm², better still 0.006 mm², and are preferably spaced apart from each other (measurement along a straight line between the barycenters of the orifices) by a distance of more than 1 mm.

In another variant, several dispensing orifices are formed entirely in the engaging part. The orifices may be constructed in such a way that the jet exiting from each orifice swirls, notably by virtue of at least two swirl channels oriented tangentially around the axis of the orifice. The engaging part may have a U-shaped axial half-section. The body may have two concentric mounting skirts between which the engaging part is fastened. The body may include a crown into which the engaging part is inserted, the crown possibly bearing one or more reliefs defining, with the engaging part, channels, notably swirl channels, for supplying the dispensing orifice.

The body may define a housing that receives the engaging part, which is then called a core.

The dispensing orifice(s) may be open at rest. The term “at rest” should be understood as meaning before the engaging part is exposed to the pressure of the product to be dispensed. Thus, in this case, the dispensing orifice(s) are already formed and open when the product is sent into the dispensing means to be dispensed. Alternatively, the dispensing orifice is formed as the product is dispensed, for example by virtue of the elasticity of at least a portion of the body or engaging part, which deforms under the pressure of the product as it is dispensed.

By means of the invention, in the case of spraying, the spray can be emitted with a relatively large flow rate, if so desired, while at the same time having a dispensing means of relatively simple construction and reliable operation. In particular, the dispensing orifice can be made with well-defined dimensions. In addition, the dispensing means may be esthetically appealing to the consumer.

The body may have a first surface that flares toward the outside, or converges toward the outside, and the engaging part may have a second surface, opposite the first surface, that diverges toward the outside, or converges toward the outside. The first surface may be conical. The second surface may be conical, at the same angle as the first surface, or at a higher or lower angle.

A different angle resulting in narrowing of the gap can lead to acceleration of the jet before it exits, which may be advantageous as regards a spray.

The dispensing orifice may or may not be unique and may or may not have an annular shape. The dispensing orifice may have a constant width in the circumferential direction. The one or more dispensing orifices may be defined between two concentric surfaces of revolution, for example in the form of cylinders of revolution.

The one or more dispensing orifices may have axial symmetry, preferably rotational symmetry, in particular around the dispensing axis. The dispensing axis is defined by the general direction toward which the product is dispensed by the dispensing means.

When the dispensing means comprises several dispensing orifices, they preferably have the following characteristics.

Their cross section is advantageously a disc.

They are preferably cylindrical in shape or approximately cylindrical in shape.

The depth of each orifice is advantageously between 0.5 and 2 mm. A long length makes it possible to create an individual spray with a reduced cone so as to create a tubular effect with a large number of orifices. A shorter length allows very wide individual spraying and further expands the application area of the multi-orifice diffuser.

The sum of the cross sections of the orifices in the ring is preferably chosen to be close to the surface area of the orifice in the nozzle.

With the same valve, it is possible to obtain various types of spray by choosing the number and the cross section of the orifices. It is possible, for example, to use a dispensing means according to the invention equipped with 80 orifices of 0.005 mm² to obtain a soft mist or a dispensing means according to the invention equipped with 10 orifices of 0.1 mm² to obtain a powerful spray.

The orifices may be distributed in various ways. They may be equidistant on the periphery of the ring, equidistant to each other on a portion of the ring, distributed in equidistant groups composed of several equidistant orifices.

It is possible to create a ring fully supporting the dispensing orifices which may be cylindrical. In this configuration, small vortex orifices can be created with a different design of the inner and outer rings to allow the creation of a ring that will have the task of creating the “centerpost” function at the back.

The engaging part is preferably fitted, thereby making it, and the body, easier to manufacture. Alternatively, the engaging part is molded as one part with the body, notably in the case of the dispensing of a foam, it then being possible for the dispensing orifice to have a larger section than in the case of the spraying of a spray.

The space formed between the body and the engaging part is fed by at least one supply channel, the cross-section of which is preferably larger than that of the dispensing orifice, thus facilitating the filling of this space before the product exits through the dispensing orifice.

A product distribution chamber may advantageously be formed, between the engaging part and the body, upstream of the dispensing orifice. This can notably facilitate the emission of a homogeneous spray.

The product supply channel can open into this chamber, which is preferably annular-shaped. Its width, which corresponds to the gap between the engaging part and the body, is preferably greater than the maximum width of the passage, via which the distribution chamber communicates with the dispensing orifice.

At least one of the body and the engaging part, preferably the body, may have at least one relief for centering the engaging part in relation to the body, and preferably at least ten, better still at least twenty, and even better still at least forty reliefs. These reliefs may extend as far as the edge of the part in which they are produced so as to generate a multitude of orifices via which jets of product exit, the centering reliefs notably being oriented parallel to the dispensing axis or obliquely in the same circumferential direction around the axis, and optionally also possibly defining, between each other, sectional narrowings that cause the jet of product to be accelerated. This or these relief(s) are preferably located in recess relative to the dispensing orifice when it is desired to generate a spray in the form of a single jet. The reliefs can be produced on the body, being for example in the form of axial ribs that are distributed regularly around the entire surface of the body opposite the engaging part.

The centring reliefs may optionally ensure alone that the engaging part is held on the body. Alternatively, the engaging part is fixed to the body somewhere other than in the region of the centering reliefs, it being possible in this case for the centering reliefs to have or not have a function of holding the engaging part on the body.

Preferably, the engaging part is fixed in relation to the body. Alternatively, the engaging part is fixed in an adjustable manner relative to the body, for example to allow the user to adjust the width of the dispensing orifice or to close it when not in use, for example by screwing it in by a quarter turn, this screwing in being accompanied by an axial movement of the engaging part relative to the body.

The engaging part may lie flush with the front end of the body so as to generate a spray with an axis substantially parallel to the axis of the engaging part.

The engaging part may extend axially beyond the front end of the body by an amount ranging between 0.01 and 1 mm, and better still between 0.01 and 0.5 mm. The spray may then diverge away from the axis of the engaging part.

The engaging part may be axially recessed from the front end of the body by an amount ranging between 0.01 and 1 mm, preferably between 0.01 and 0.5 mm. The spray can then be convergent toward the axis of the engaging part.

The invention makes it easy to make a dispensing orifice with a circular inner contour, if so desired. The inside diameter of the passage formed through the dispensing means is, for example, greater than or equal to 10 mm, better still 15 mm, 20 or 30 mm. When the passage is not circular in cross-section, the “inside diameter” refers to that of the largest circle inscribed in this passage.

The dispensing means may include at least two housings and two engaging parts located in the housings and each defining with the body, at rest, a dispensing orifice according to the invention. The dispensing axes may then be parallel or not parallel, intersecting or not intersecting, for example converging toward each other.

The dispensing orifice may have, in axial half section, an axis that converges or diverges in relation to the spraying direction.

The invention also relates to an aerosol device comprising:
a container containing:
a composition comprising an amount of greater than or equal to 30% by weight, relative to the total weight of the composition, of one or more C6-C14 branched alkanes, one or more fatty substances other than the C6-C14 branched alkanes and one or more propellants,
a means for dispensing said composition comprising:
   a body,
   an engaging part, in particular a core, defining with the body, at rest, at least one dispensing orifice having an annular cross section.

The invention also relates to an aerosol device comprising:
a container including a valve stem or pump stem, containing:
   a composition comprising an amount of greater than or equal to 30% by weight, relative to the total weight of the composition, of one or more C6-C14 branched alkanes, one or more fatty substances other than the C6-C14 branched alkanes and one or more propellants,
a means for dispensing said composition comprising:
   a body provided with an end piece for connecting to the valve stem or pump stem,
   a part attached to the body, defining at least partially a dispensing orifice of notably annular cross-section at rest or several dispensing orifices distributed around a dispensing axis (Z),
   the dispensing means not passing through along the dispensing axis (Z),
   the body being closed along the dispensing axis (Z) and said part notably being annular-shaped, or
   the body having a through opening along the dispensing axis (Z) and said part closing off said opening.

The invention may be understood more clearly on reading the following detailed description of nonlimiting implementation examples thereof and on examining the appended drawing, in which:

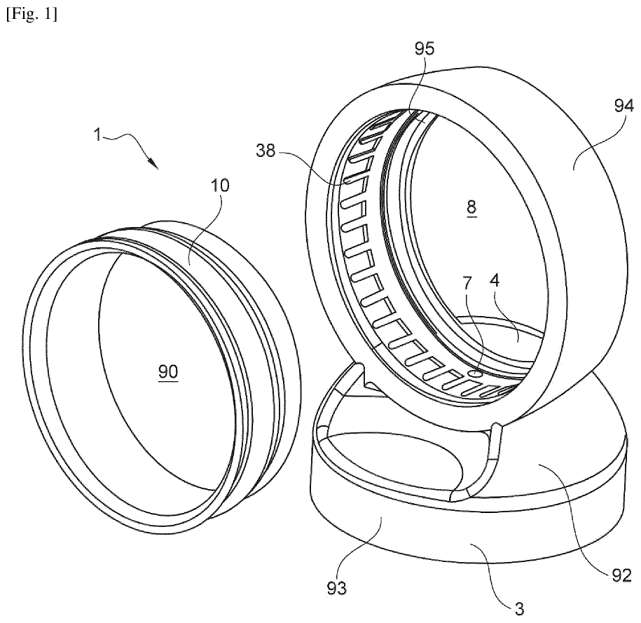

FIG. 1 represents, schematically in perspective, an example of a dispensing means made in accordance with the invention, before mounting the engaging part on the body of the dispensing means,

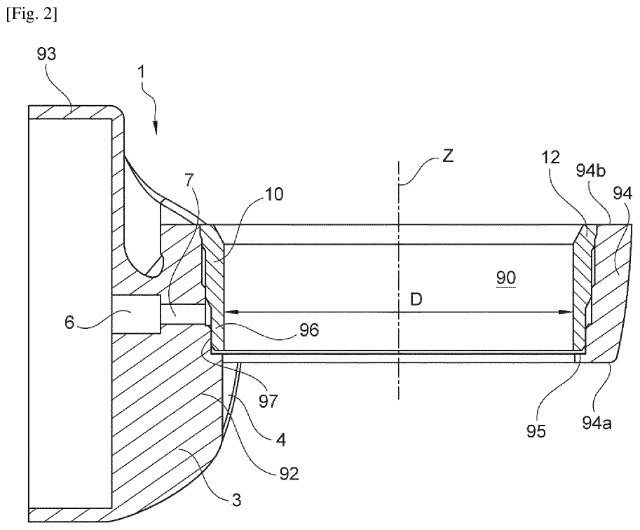

FIG. 2 shows the dispensing means after the engaging part has been fitted in the body,

FIG. 3 is a view similar to FIG. 1 in partial section,

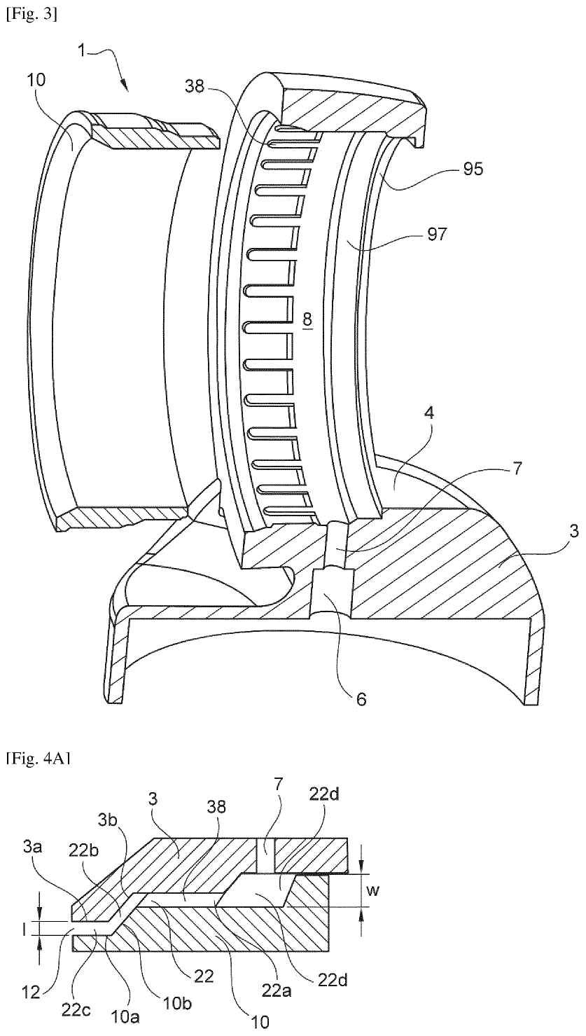

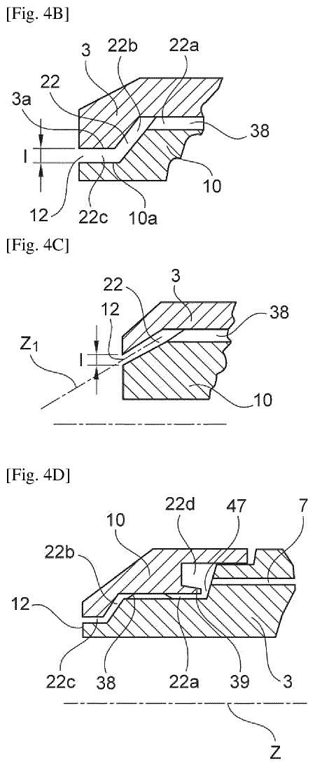

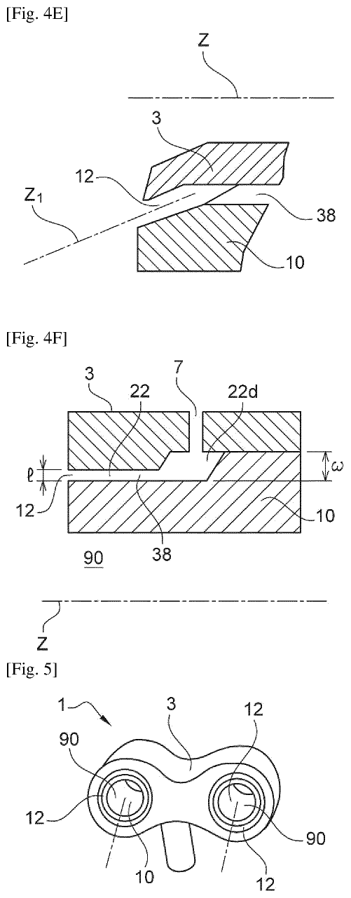

FIGS. 4A to 4F illustrate different arrangements, among others, of the engaging part and the body,

FIG. 5 illustrates the possibility of making the dispensing means with two dispensing orifices according to the invention,

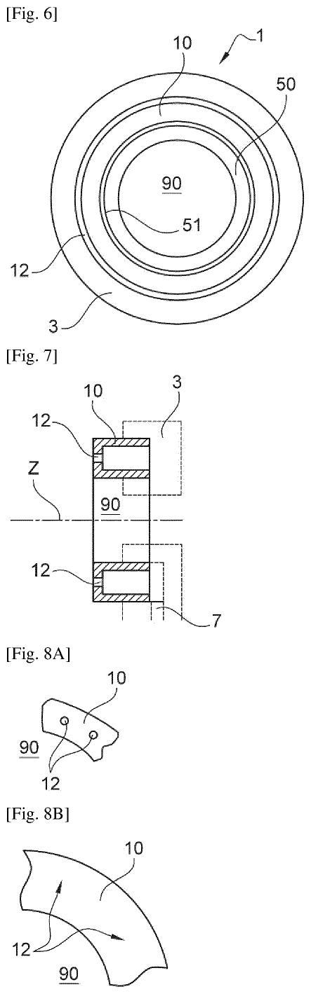

FIG. 6 shows a front view of a dispensing means having concentric dispensing orifices,

FIG. 7 is an axial cross section of an alternative embodiment of the engaging part,

FIGS. 8A and 8B are partial front views of different examples of configurations of the engaging part of FIG. 7,

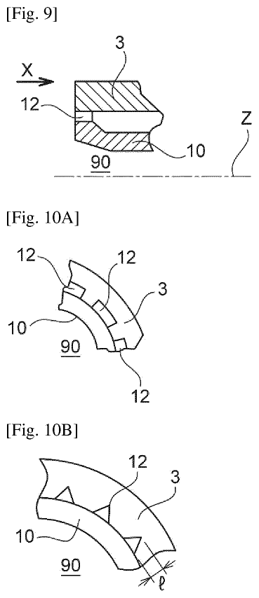

FIG. 9 is a partial axial cross section of an alternative embodiment of the dispensing orifice,

FIGS. 10A and 10B are front views along X of different examples of configurations according to FIG. 9,

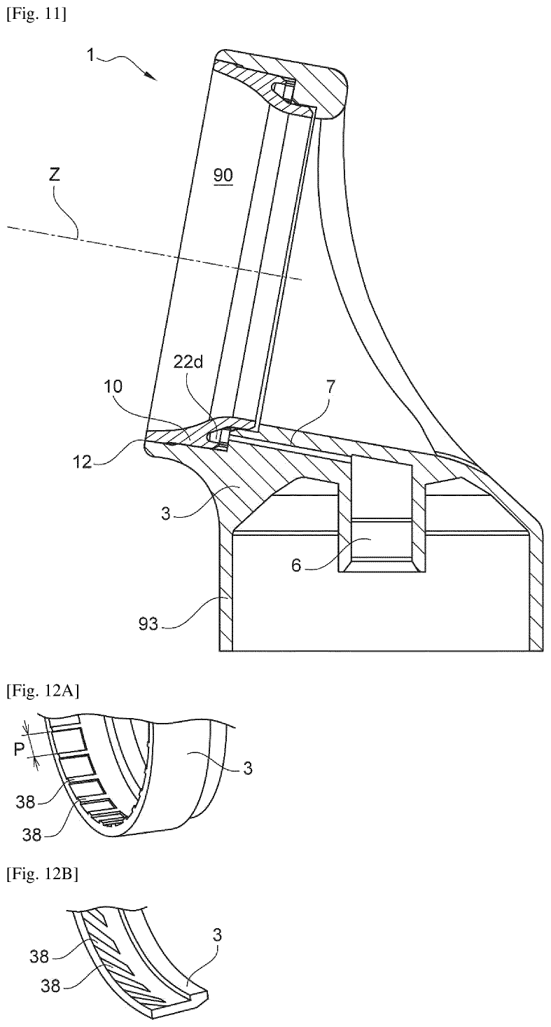

FIG. 11 is a view similar to FIG. 2 of an alternative embodiment of the dispensing means,

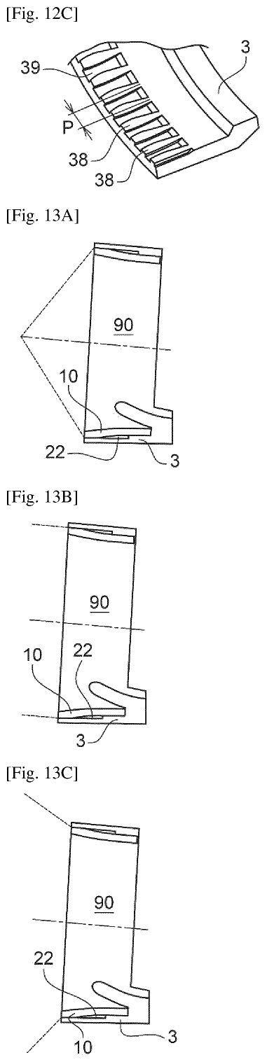

FIGS. 12A to 12C illustrate different examples of arrangements of the reliefs on the body,

FIGS. 13A to 13C illustrate various examples of configurations of the engaging part relative to the body,

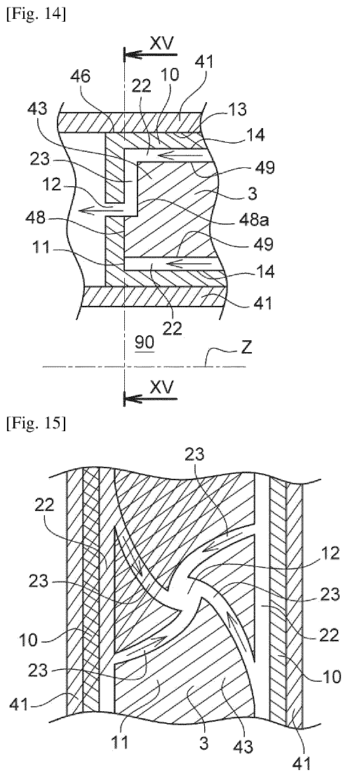

FIG. 14 is a partial axial cross section of an alternative embodiment of the dispensing orifice,

FIG. 15 is a section along XV of FIG. 14,

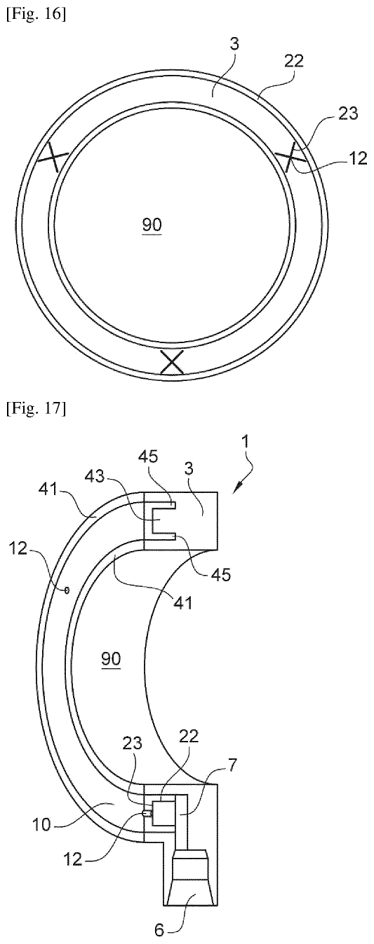

FIG. 16 is an exemplary embodiment of the body according to FIG. 14, and

FIG. 17 is a cutaway perspective view of an example of a dispensing means according to the configuration of FIG. 14.

In the drawing, the actual respective proportions of the various constituent elements have not always been respected, for the sake of clarity.

The dispensing means 1 shown in FIGS. 1 to 3 is intended to equip a container not shown, provided with a hollow valve stem or pump stem, through which the product to be dispensed contained in the container is conveyed to the dispensing means 1.

The container may notably be a pressurized container of the aerosol can type, containing a propellant gas, for instance compressed air or a liquefied gas.

The container may be equipped with a valve and the valve may be opened, for example, by pressing the hollow stem or alternatively by tilting the latter. When the container is equipped with a pump, the pump may be actuated, for example, by pressing the hollow stem along its longitudinal axis.

The dispensing means 1 includes a body 3, which can be made as one block by molding in a single piece or include several elements made separately and assembled.

The dispensing means 1 may include, as can be seen in FIG. 2, a housing 6 intended to engage with the hollow stem to allow the product delivered by the latter to reach a supply channel 7 which opens into a housing 8 of the body 3. The housing 6 has a dimension adapted to the outside diameter of the stem, so as to obtain a leaktight fit of the stem in the housing 6, so that the product delivered through the stem passes entirely into the supply channel 7. The latter is, for example, coaxial with the stem of the container, but could be oriented in some other way and include, for example, a plurality of differently oriented portions.

An engaging part 10, called core hereinbelow when it is inside the body, is fixed in the housing 8 and defines, for example, with the body 3 a dispensing orifice 12 of annular cross section, as illustrated.

For the purposes of the present invention, the term “annular cross section” should be understood as meaning any cross section that follows a closed contour, whether this contour is circular, elliptical, polygonal or some other shape.

Passing axially through the core 10 is an opening 90, the inside diameter D of which may be relatively large, for example greater than or equal to 10 mm, better still 15, 20 or 30 mm.

The opening 90 helps to give the dispensing means a particularly esthetic appearance. In addition, the opening 90 may allow air to circulate through the dispensing means under the entrainment effect of a spray emitted from the dispensing orifice 12. This may help to increase the range of the spray and may increase the freshness effect provided by the spray, where appropriate.

The opening 90 may also allow a finger or a lock of hair to be inserted through the dispensing means, which may allow a product to be applied to the entire circumference of the item inserted through the dispensing means in one motion. This may be an advantage for applying, for example, an antiseptic or care product to a finger or for treating a lock of hair.

The dispensing axis Z may be perpendicular to the longitudinal axis X of the container on which the dispensing means is mounted, as illustrated.

The dispensing means 1 includes a base 92 that defines a surface 4 that can be pressed by the user to effect dispensing.

The base 92 may be extended at the bottom by a trim skirt 93, which covers the upper portion of the container.

The housing 8 that receives the core 10 is defined by a crown 94 of axis Z, the lower side of which joins the base 92. The supply channel 7 passes through the base 92 and ends in the housing 8 at a distance from the axial ends, along the Z axis, of the ring 94, preferably being closer to the rear end 94a than to the front end 94b, as can be seen in FIG. 2.

The body 3 may have, as illustrated, a shoulder 95 close to the rear end 94a, against which the core 10 may come into axial abutment, where appropriate, on completion of its assembly.

The core 10 and the housing 8 may have annular surfaces 96 and 97, in leaktight contact, to close the space formed between the core 10 and the body 3 behind the supply channel 7.

Preferably, the circumferential width I of the dispensing orifice 12 about the spraying direction Z is constant. It is not a departure from the scope of the present invention when this width I varies, for example, so as to take into account the possibly nonuniform pressure drop experienced by the product flow upstream of the dispensing orifice 12. This nonuniform pressure drop results, for example, from the geometry of the space between the core and the body, particularly the presence of corners or crossings. By varying the width I, it can be made easier for the product to exit where this pressure drop is the greatest, if it is sought to have as uniform a spray as possible.

The width I of the dispensing orifice is, for example, between 0.01 and 2 mm.

The core 10 can be attached to the body 3 in various ways. In the example illustrated in FIGS. 1 to 3, the core 10 is retained on the body 3 by friction.

In the example illustrated, the core 10 is produced separately from the body 3 and is attached to the latter. The core 10 can be produced from the same thermoplastic material as the body 3 or alternatively from a different thermoplastic material. It is also possible to use a metal material to produce the core 10.

Axial ribs 38 are formed on the internal circumference of the housing 8, as can be seen in particular in FIGS. 1 and 3, so as to center the core 10 in the housing 8. The centering reliefs 38 may be, as illustrated in FIGS. 12A to 12C, parallel or oblique in the direction circumferential to the axis Z, or curved. Each relief 38 may have, when seen in a top view, a contour that is polygonal, notably rectangular or trapezoidal, or that has a shape that is flared in the direction of the dispensing edge. Two centering reliefs 38 may define, between each other, a narrowing 39 in the vicinity of the dispensing orifice so as to accelerate the fluid via the Venturi effect. The number of centering reliefs 38 is preferably at least 10, better still 20, even better still 40.

The space 22 formed between the core 10 and the body 3 may have the configuration illustrated schematically in FIG. 4A, and may open onto the dispensing orifice 12 via an annular terminal portion 22c formed between two surfaces 3a and 10a which are in the form of circular cylinders about the axis Z.

The terminal wall 22c is attached to a proximal portion 22a via an inclined intermediate portion 22b formed between opposite surfaces 3b and 10b.

The centering reliefs 38 extend in the proximal portion 22a. The latter is fed with product via the distribution chamber 22d.

When the user actuates the dispensing means 1, the product enters the space 22 between the core 10 and the body 3 via the supply channel 7 and can be dispensed as a spray through the dispensing orifice 12.

In the example in FIGS. 1 to 3, the spray is continued angularly around the dispensing axis on account of the absence of contact between the core 10 and the body 3 in the region of the dispensing orifice 12. Specifically, the bearing region or regions between the core 10 and the body 3 are for example located, as illustrated, set back from the dispensing orifice 12 by a distance (measured along the dispensing axis Z) of at least 0.5 mm.

The spray may be discontinuous angularly around the dispensing axis on account of the presence, in particular at the reliefs 38, of contact between the core 10 and the body 3 at the product outlet.

Preferably, the cross-sectional area of the supply channel 7 is greater than the cross-sectional area of the dispensing orifice 12, so that the space upstream of the dispensing orifice can be quickly filled with the product, which may contribute toward the formation of a homogeneous spray from the start of spraying.

The distribution chamber 22d formed upstream of the space 22a in which the centering reliefs 38 extend receives the product delivered through the supply channel 7.

The width w of the distribution chamber 22d is greater than the width I of the end portion 22c that opens into the dispensing orifice 12.

The distribution chamber 22d improves the dispensing of product before the product reaches the narrower portions of the passage via which the product discharges.

FIGS. 4B and 4C illustrate various other examples of possible configurations for the space 22 formed between the core 10 and the body 3 for product circulation to the dispensing orifice.

In the example in FIG. 4B, the space 22 formed between the core and the body comprises a proximal portion 22a in which the centering reliefs 38 of the core 10 extend in relation to the body 3, extended by an intermediate portion 22b which forms an angle with the spraying direction Z, for example a re-entrant angle. This intermediate portion 22b can be connected to an end portion 22c, which opens onto the dispensing orifice 12, this end portion being for example, as illustrated, defined between two surfaces 3a and 10a, which are circularly cylindrical, parallel to the dispensing direction Z. The variant in FIG. 4B does not include a distribution chamber.

In the variant of FIG. 4C, the terminal portion 22c engages directly with that portion 22a in which the centering reliefs 38 extend. The terminal portion 22c forms, for example, an angle with the dispensing direction Z. Thus, in axial half-section, the axis Z1 of the orifice 12 is, for example, convergent, as illustrated.

In the variant shown in FIG. 4D, the engaging part 10 is external to the body 3. The engaging part 10 is fixed to the body 3 so as to provide with the body 3 the distribution chamber 22d, facing the supply channel 7. The portions 22a, 22b and 22c allow the product to be conveyed to the dispensing orifice 12.

The supply channel 7 opens into the distribution chamber 22d, for example, by a portion oriented parallel to the dispensing axis Z.

Centring reliefs 38 are produced for example on the body 3. The engaging part 10 can be produced, as illustrated, with an annular lip 39 which partially delimits the distribution chamber 22d and forms a narrowing 47 of the section between the chamber 22d and the portion 22a.

FIG. 4E illustrated the possibility of having an angle between the axial half-section axis Z2 of the orifice 12 and the dispensing axis, which is divergent.

In the variant in FIG. 4F, the possibility of having no angle between the dispensing axis and the axis Z of the engaging part 10 is illustrated. The supply channel 7 opens, for example, onto a distribution chamber 22d. The product is conveyed toward the dispensing orifice 12 via channels 22 including reliefs 38. The reliefs 38 extend as far as the edge of the dispensing orifice 12 and define a plurality of orifices for delivering the product in the form of a plurality of jets.

The invention is not limited to a dispensing head including a single dispensing orifice 12 made in accordance with the invention.

By way of example, FIG. 5 illustrates a dispensing head 1 which includes two dispensing orifices 12.

When there are a plurality of dispensing orifices, these may be distributed in a multitude of ways on the dispensing means. For example, the spraying axes are parallel, or form an angle, in that, for example, they intersect.

It has been illustrated in FIGS. 7, 8A and 8B that the dispensing means may have a plurality of dispensing orifices 12 formed entirely within the core 10 so as to dispense the product in the form of a plurality of jets, for example. The dispensing orifices 12 may have many shapes when viewed along their transverse axis, notably circular or triangular, as illustrated in FIGS. 8A and 8B. The dispensing orifices 12 may be drilled into the core 10, for example by laser drilling.

The core 10 may have a U-shaped axial half-section, as illustrated in FIG. 7. The body 3 may comprise two concentric mounting skirts 41 which define between them a space for mounting the core 10, and may include, at its center, a crown 43 serving to support the engaging part 10. The skirts 41 define with the crown 43 two annular channels 45 in which the arms of the U are placed. For each orifice 12, the crown 43 can comprise two channels 22 for the supply of liquid to this orifice 12.

During mounting, as illustrated in FIGS. 14 and 17, the core 10 may bear against the pad 43, the end face 48 of the crown 43 making contact with the inner face 11 of the core 10. The arms of the U of the core 10 are fixed in the channels 45, the internal face 46 of the mounting skirts 41 being in contact with the face 13 of the core 10. The inner faces 14 of the arms of the U and the side surfaces 49 of the crown 43 may define, between each other, the channels 22 for supplying liquid to the dispensing orifice 12. The crown 43 may have, notably in the form of impressions, on its outer face 48, supply channels 23 allowing the liquid to pass from the supply channels 22 to the dispensing orifice 12.

The supply channels 22 open, upstream of the dispensing orifices 12, onto the supply channels 23, which lead to the dispensing orifice 12. The supply channels 23 generate, via their orientation relative to the dispensing orifice, a swirling flow at the outlet of the dispensing orifice 12. This configuration is more particularly useful in the case of a non-liquefied carrier gas.

In one variant, the supply channels 22 may be made in the form of impressions on the side surface 49 of the body and/or on the inner faces 14 of the core 10.

In a variant not shown, the core 10 possesses, notably in the form of impressions on its inner face 11, supply channels 23, the end face 48 of the crown 43 possibly being smooth.

In one variant, the crown 43 is not circumferentially continuous and defines pads. The pads are placed upstream of the dispensing orifices 12 and may have, upstream of the dispensing orifices 12, supply channels 22 and 23 as described above.

In the variant of FIGS. 4F, 9 and 10, the dispensing orifices 12 are formed between the core 10 and the body 3, being distributed, for example, all around the spray axis Z. The core 10 or the body 3 may have centering reliefs 38 which circumferentially delimit the dispensing orifices 12. The centering reliefs 38 may, as illustrated in FIGS. 12A to 12C, extend as far as the edge of the core 10 over its entire periphery and define, between each other, dispensing orifices 12. The number of dispensing orifices 12 is preferably at least 10, better still 20, even better still 40. The cross section of a dispensing orifice 12 is, for example, greater than 0.003 mm². The dispensing orifices 12 are preferably spaced apart by a space of at least 1 mm, which is the same as the pitch p between the centering reliefs. As illustrated in FIGS. 10A and 10B, the dispensing orifices 12 may have a polygonal cross section, notably a triangular cross section.

The core 10 may, as illustrated in FIG. 13A, be recessed relative to the body by a value of between 0.01 and 1 mm, better still between 0.01 and 0.5 mm. The body 3 protrudes from the dispensing orifice and can generate a convergent spray.

The core 10 may, as illustrated in FIG. 13B, be flush with the body 3. The spray can then be straight.

The core 10 may extend, as illustrated in FIG. 13C, forward, relative to the body 3 by an amount ranging between 0 and 1 mm, better still between 0 and 0.5 mm. The spray can then be divergent.

If an additional dispensing orifice is provided, for example by attaching inside the core 10 a second core 50 which defines with the first core 10 a second dispensing orifice 51 which is coaxial with the first dispensing orifice, as illustrated in FIG. 6, this does not depart from the scope of the present invention. A passage 90 continues to be formed through the dispensing means.

The dispensing orifice may be fed with more than one product.

The dispensing means may be fed with two products which are dispensed through separate dispensing orifices.

The axis Z may be non-perpendicular to the axis of the stem of the container on which the dispensing means is mounted, as shown in FIG. 11. In this example, the axis Z is oriented upward when the container is vertical with the dispensing means at the top.

The supply channel 7 can be oriented substantially parallel to the dispensing axis Z, at least as regards the portion which opens out facing the engaging part 10. The latter may be produced with an annular lip 39 which defines a narrowing of section 47.

The configuration may be similar to that in FIG. 4D apart from the fact that the engaging part 10 is outside the body 3 in the example in FIG. 4D and inside it in the example in FIG. 11.

The dispensing means may be arranged to allow the mounting of a protective cover and may include, where appropriate, an on/off system to prevent actuation of the device at a certain position of the dispensing means relative to the container or at a certain position of a locking element of the dispensing means relative thereto.

In non-illustrated variants, the dispensing orifice is formed between a body and a engaging part, the body being radially internal relative to the engaging part, the product supply channel passing through the body. All of the features described with reference to the figures can be found in such variants in which the body is radially internal relative to the engaging part.

The example that follows serves to illustrate the invention.

### EXAMPLE

In the examples that follow, all the amounts are indicated as weight percentage of product as active materials relative to the total weight of the composition.

### Example 1

The composition below was prepared from the compounds indicated in the table below.

TABLE 1

| | A |
|---|---|
| C12-15 Alkyl benzoate | 1.7 |
| Dimethiconol at 15% AM in | 1.3 (0.195 dimethiconol |
| isododecane | and 1.105 isododecane) |
| Isododecane | 89.2 |
| Fragrance | 0.7 |

Composition A was introduced into two devices D1 and D2:

The aerosol container comprises 50% by weight of composition A and 50% by weight of

isobutane.

TABLE 2

| D1 comparative | D2 invention |
|---|---|
| Pump-action bottle | Valve: Coster GI -internal nozzle- 0.32 mm/RI - |
| Aptar PZ2 DL 190 μl | internal restriction- 0.40 mm |
| pump with DU3020 | Dispensing means comprising 10 orifices of 0.05 |
| nozzle | mm unit section distributed on the annular surface |

Composition A is applied to dry hair via these two devices on locks of natural hair weighing 2.7 g, at a rate of 0.2 g per lock.
The amount of composition A deposited on each lock is identical with the two devices.

### Application Quality

The quality of application was evaluated by five experts, on dry hair, in a blind test. Each of the five experts rated each lock, giving “2 points” to the best performing side, “0 points” to the worst performing side and “1 point” in the event of a tie.
To evaluate the quality of the application, each expert observed the lock just after application of the composition, notably evaluating whether the dispensing and size of the droplets present on the fibers is homogeneous.

TABLE 3

Application quality

| | D1 Comparative | D2 invention |
|---|---|---|
| Expert 1 | 0 | 2 |
| Expert 2 | 0 | 2 |
| Expert 3 | 0 | 2 |
| Expert 4 | 0 | 2 |
| Expert 5 | 0 | 2 |
| Mean | 0 | 2 |

100% of the experts (5 out of 5) judged that the device D2 according to the invention made it possible to obtain an extremely fine and light deposit, homogeneously dispensed on the locks of hair.
Natural Feel, without Transfer-Natural Aspect, without a “Wet Effect” Look
The feel and look were evaluated by five experts, on dry hair, during a blind test. Each of the five experts rated each lock, giving “2 points” to the best performing side, “0 points” to the worst performing side and “1 point” in the event of a tie.
To evaluate the feel, the expert takes the lock between thumb and index finger, descends from the top of the lock to the ends and at the same time effects a gentle oscillating movement with the thumb to get a good feel for the presence of any deposit. The evaluation of the unnatural feel is also accompanied by the observation/sensation of product transfer onto the fingers.
The scores attributed by the experts are given below:

TABLE 4

| | | | Visual appearance | |
|---|---|---|---|---|
| | Transfer-free feel | | Non-greasy, no wet effect | |
| | D1 | D2 | D1 | D2 |
| | Comparative | invention | Comparative | invention |
| Expert 1 | 0 | 2 | 0 | 2 |
| Expert 2 | 0 | 2 | 0 | 2 |
| Expert 3 | 0 | 2 | 0 | 2 |
| Expert 4 | 0 | 2 | 0 | 2 |
| Expert 5 | 0 | 2 | 0 | 2 |
| Mean | 0 | 2 | 0 | 2 |

100% of the experts (5 out of 5) judged that the device D2 according to the invention made it possible to obtain a more natural feel and look, i.e. without transfer, non greasy and wet appearance, than the comparative device D1.

### Example 2

The following compositions were prepared from the compounds indicated in the table below.

TABLE 5

| | A invention | B comparative |
|---|---|---|
| C12-15 Alkyl benzoate | 0.85 | 0.85 |
| Fragrance/perfume | 0.35 | 0.35 |
| Isododecane | 48.17 | 28.54 |
| Isohexadecane | — | 19.63 |
| Dimethiconol | 0.63 | 0.63 |
| Isobutane | 50 | 50 |

Each of the compositions was introduced into an aerosol device equipped with a diffuser according to the invention comprising a Coster valve with an internal nozzle of 0.32 mm, an internal restriction of 0.40 mm and a dispensing means according to the invention comprising 10 orifices of 0.05 mm unit cross section distributed on the annular surface.
Compositions A and B are applied to locks of natural hair weighing 2.7 g, at a rate of 0.2 g per lock.
The application is done as a finish on dry hair.
The amount of composition A deposited on each lock is identical with the two devices.
Natural Feel, without Transfer-Natural Aspect, without a “Wet Effect” Look
The feel and look were evaluated by five experts, on dry hair, during a blind test. Each of the five experts rated each lock, giving “2 points” to the best performing side, “0 points” to the worst performing side and “1 point” in the event of a tie.
To evaluate the feel, the expert takes the lock between thumb and index finger, descends from the top of the lock to the ends and at the same time effects a gentle oscillating movement with the thumb to get a good feel for the presence of any deposit.
The evaluation of the unnatural feel is also accompanied by the observation/sensation of product transfer onto the fingers.
The scores attributed by the experts are given below:

TABLE 6

| | Feel | | Visual appearance | |
|---|---|---|---|---|
| | B | A | B | A |
| | Comparative | Invention | Comparative | Invention |
| Expert 1 | 0 | 2 | 0 | 2 |
| Expert 2 | 0 | 2 | 0 | 2 |
| Expert 3 | 0 | 2 | 0 | 2 |
| Expert 4 | 0 | 2 | 0 | 2 |
| Expert 5 | 0 | 2 | 0 | 2 |
| Mean | 0 | 2 | 0 | 2 |

100% of the experts (5 out of 5) judged that composition A according to the invention made it possible to obtain a more natural feel and look without transfer, non-greasy and without a wet effect than composition B comprising less than 30% by weight of C6-C14 branched alkanes.

### Example 3

The composition below was prepared from the compounds indicated in the table below.

TABLE 7

| | A invention |
|---|---|
| C12-15 Alkyl benzoate | 0.85 |
| Fragrance/perfume | 0.35 |
| Isododecane | 48.17 |
| Dimethiconol | 0.63 |
| Isobutane | 50 |

The composition was introduced into an aerosol device equipped with a diffuser according to the invention comprising a Coster valve with an internal nozzle of 0.32 mm, an internal restriction of 0.40 mm and a dispensing means according to the invention comprising 10 orifices of 0.05 mm unit cross section distributed on the annular surface.
The drying time of 10 locks moistened with water and then treated with the composition according to the invention was compared to 10 untreated locks moistened with water.
The locks were weighed before and after moistening.
The locks were dried with a dryer positioned 10 cm from each of the locks. Drying was performed in 10-second periods until the weight of each lock no longer varied.

TABLE 8

| | A invention | Control |
|---|---|---|
| Drying time in seconds | 74 | 46 |

The process according to the invention made it possible to reduce the drying time by more than 70% compared to an untreated lock.

### Example 4

The following compositions were prepared from the compounds indicated in the table below.

TABLE 9

| | D1 | D2 | D3 |
|---|---|---|---|
| | Comparative | Invention | Invention |
| VP/VA copolymer | 0.01 | 0.01 | 0.01 |
| Mineral oil paraffinum liquidum | 0.48 | 0.48 | 0.48 |
| Isopropyl palmitate | 4.80 | 4.80 | 4.80 |
| Dimethicone | 4.50 | 4.50 | 4.50 |
| Octyldodecanol | 1.02 | 1.02 | 1.02 |
| Fragrance | 0.21 | 0.21 | 0.21 |
| Isododecane | — | 30 | — |
| Isohexadecane | — | — | 30 |
| Butane | 40.00 | 40.00 | 40.00 |
| Ethanol | qs100 | qs100 | qs100 |

Each of the compositions D1, D2 and D3 was introduced into an aerosol device equipped with a diffuser according to the invention comprising a Coster valve with an internal nozzle of 0.32 mm, an internal restriction of 0.40 mm and a dispensing means according to the invention comprising 10 orifices of 0.05 mm unit cross-section distributed on the annular surface.
The compositions D1, D2 and D3 were applied in a standardized manner to a malleable head of natural straight hair previously combed and separated into three parts (locks), at a rate of 0.8 g per application (two return trips from top to bottom).
The locks were evaluated after one minute of drying.
The sheen was evaluated visually by nine experts in a blind test. They looked at the head of hair and estimated the reflection of light at the hair curve.
Each of the nine experts rated each of the three locks using the following scale:
(very good)=2 points
(good)=1 point
(poor)=0 points

TABLE 10

| | D1 | D2 | D3 |
|---|---|---|---|
| | Comparative | invention | invention |
| Expert 1 | 0 | 2 | 1 |
| Expert 2 | 0 | 1 | 2 |
| Expert 3 | 0 | 1 | 2 |
| Expert 4 | 0 | 1 | 2 |
| Expert 5 | 0 | 2 | 1 |
| Expert 6 | 0 | 1 | 2 |
| Expert 7 | 0 | 2 | 1 |
| Expert 8 | 0 | 1 | 2 |
| Expert 9 | 0 | 1 | 2 |
| Total | 0 | 17 | 15 |

Compositions D2 and D3 according to the invention lead to improved sheen relative to composition D1 according to the prior art.

1. An aerosol device comprising:
a container containing a composition comprising an amount greater than or equal to 30% by weight relative to the total weight of the composition of one or more C6-C14 branched alkanes, one or more fatty substances other than the C6-C14 branched alkanes and one or more propellants,
a means for dispensing said composition, including:
a body (3) that is open at its two opposite axial ends,
an engaging part (10) that is open at its two opposite axial ends, at least partially defining at least one dispensing orifice (12).

2. The device as claimed in claim 1, characterized in that the C6-C14 branched alkane(s) are chosen from C8-C14 alkanes, in particular from isohexane, isooctane, isodecane, isododecane and mixtures thereof, preferably isododecane.

3. The device as claimed in either of the preceding claims, characterized in that the C6-C14 branched alkane(s) are present in an amount ranging from 30% to 70% by weight, preferably from 40% to 60% by weight and better still from 45% to 55% by weight relative to the total weight of the composition.

4. The aerosol device as claimed in any one of the preceding claims, characterized in that the fatty substance(s) other than the C6-C14 branched alkanes are chosen from nonsilicone liquid fatty substances and silicones, preferably from triglycerides of plant or synthetic origin, liquid fatty esters other than triglycerides, liquid fatty alcohols, liquid fatty ethers, dialkyl carbonates, silicones and mixtures thereof.

5. The aerosol device as claimed in the preceding claim, characterized in that the fatty substance(s) other than the C6-C14 branched alkanes are chosen from liquid fatty esters, silicones and mixtures thereof.

6. The aerosol device as claimed in either of claims 4 and 5, characterized in that the fatty substance(s) other than the C6-C14 branched alkanes are chosen from esters of aromatic acids and of C8-C18 fatty alcohols, polydimethylsiloxanes bearing dimethyl silanol (dimethiconol) end groups, and mixtures thereof.

7. The aerosol device as claimed in any one of the preceding claims, characterized in that the fatty substance(s) other than the C6-C14 branched alkanes are present in a total amount ranging from 0.1% to 15% by weight, better still from 0.5% to 10% by weight and even more preferentially from 1% to 5% by weight relative to the total weight of the composition.

8. The aerosol device as claimed in any one of the preceding claims, characterized in that the propellent(s) are chosen from air, nitrogen, carbon dioxide, dimethyl ether, volatile hydrocarbons, notably such as C₃-C₅ alkanes, chlorinated and/or fluorinated hydrocarbons such as 1,1-difluoroethane and mixtures thereof, preferably chosen from dimethyl ether and C₃-C₅ alkanes and in particular n-butane, propane and isobutane, and mixtures thereof, preferentially isobutane.

9. The aerosol device as claimed in any one of the preceding claims, characterized in that the propellant(s) are present in an amount ranging from 30% to 70% by weight, better still from 40% to 60% by weight and even more preferentially from 45% to 55% by weight relative to the total weight of the composition.

10. The aerosol device as claimed in any one of the preceding claims, characterized in that the dispensing orifice (12) is defined between the engaging part (10) and the body (3).

11. The device as claimed in any one of the preceding claims, characterized in that the dispensing orifice (12) is annular and preferably has, in the circumferential direction, a constant width (I).

12. The device as claimed in either of claims 10 and 11, characterized in that the dispensing orifice (12) is axially symmetrical, preferably rotationally symmetrical.

13. The device as claimed in any one of claims 1 to 12, characterized in that the engaging part (10) at least partially defines a plurality of dispensing orifices (12).

14. The device as claimed in the preceding claim, characterized in that the number of dispensing orifices (12) is, limits inclusive, between 2 and 80, preferably between 5 and 60.

15. The device as claimed in either of claims 13 and 14, characterized in that the dispensing orifices (12) each have a cross section of greater than or equal to 0.0025 mm².

16. A process for conditioning keratin fibers, comprising the use of the device as claimed in any one of the preceding claims.

17. The process as claimed in the preceding claim, comprising a step of applying to wet or dry hair a composition sprayed from an aerosol device as claimed in any one of claims 1 to 15, and optionally rinsing, preferably without rinsing after an optional leave-on time.
